(19) United States
(12) Patent Application Publication
(10) Pub. No.: US 2019/0084925 A1
(43) Pub. Date: Mar. 21, 2019

(54) METHOD FOR PREPARING OXYSULPHIDE AND FLUORINATED DERIVATIVES IN THE PRESENCE OF AN ORGANIC SOLVENT

(71) Applicant: RHODIA OPERATIONS, Paris (FR)

(72) Inventors: Valery DAMBRIN, SAINT GENIS-LAVAL (FR); Denis REVELANT, GENAS (FR)

(21) Appl. No.: 15/534,083

(22) PCT Filed: Dec. 7, 2015
(86) PCT No.: PCT/EP2015/078756

§ 371 Date: Jun. 8, 2017

(30) Foreign Application Priority Data
Dec. 9, 2014 (FR) 1462098

(51) Int. Cl. C07C 303/32; C07C 313/04; C07C 303/22; C07C 303/00

(52) U.S. Cl. CPC C07C 303/32; C07C 303/00; C07C 303/22; C07C 313/04

(57) ABSTRACT

The present invention concerns a method for preparing an oxysulphide and fluorinated derivative of formula (III) Ea-SO₃R (III) that comprises bringing a compound of formula (II) Ea-SOOR (II)—Ea representing the fluorine atom or a group having 1 to 10 carbon atoms chosen from the fluoroalkyls, the perfluoroalkyls and the fluoroalkenyls; and—R representing hydrogen, a monovalent cation or an alkyl group; into contact, in the presence of a polar aprotic organic solvent, with an oxidising agent.

A subject of the present invention is a novel process for the preparation of oxysulfide and fluorinated derivatives, employing an oxidation reaction in the presence of an organic solvent.

The invention more particularly targets the preparation of perfluoroalkanesulfonic acids, in particular trifluoromethanesulfonic acid.

Perhaloalkanesulfonic acids, and more particularly trifluoromethanesulfonic acid, better known as “triflic acid”, are used as catalysts or as intermediates in organic synthesis.

A current route for the industrial synthesis of trifluoromethanesulfonic acid employs two mains steps. Firstly, an alkali metal salt, generally a potassium salt, of trifluoromethanesulfinic acid, is synthesized by sulfination reaction starting from a salt of trifluoromethanecarboxylic acid, in an organic aprotic solvent, typically N,N-dimethylformamide (DMF). Secondly, the salt of trifluoromethanesulfinic acid is oxidized in aqueous medium, generally by aqueous hydrogen peroxide, to give a salt of trifluoromethanesulfonic acid, which, after acidification, will give triflic acid. The preparation of triflic acid is described for example in documents EP 0 396 458 and EP 0 735 023.

Even though this process is generally satisfactory, some elements could be improved. Firstly, it is desirable to limit the steps of switching between organic medium/aqueous medium between the sulfination and oxidation reactions, since these switching steps may be complex to carry out. In addition, the presence of water during the acidification step is a drawback, and means must be employed to capture this residual water; typically, the addition of sulfuric anhydride (SO₃). The addition of sulfuric anhydride to capture the residual water unfortunately results in the generation of a large amount of sulfuric effluents.

The present invention aims to propose a novel process for the preparation of oxysulfide and fluorinated derivatives, which are in particular of use in the synthesis of trifluoromethanesulfonic acid, and which do not have the abovementioned drawbacks.

More specifically, according to a first aspect thereof, the present invention relates to a process for the preparation of an oxysulfide and fluorinated derivative of formula (III)

Ea-SO₃R (III)

comprising bringing into contact, in the presence of an organic polar aprotic solvent, a compound of formula (II)

Ea-SOOR (II)
Ea representing a fluorine atom or a group having from 1 to 10 carbon atoms, selected from fluoroalkyls, perfluoroalkyls and fluoroalkenyls; and
R representing hydrogen, a monovalent cation or an alkyl group;
   with an oxidizing agent.

Surprisingly, the inventors have shown that the oxidation could be carried out in an organic solvent in order to give rise to the desired oxysulfide and fluorinated derivative, in particular to potassium trifluoromethanesulfonate, with performance levels in terms of kinetics and selectivity which are at least identical to the performance levels of an oxidation in aqueous solvent.

In order to give rise, for example, to potassium trifluoromethanesulfonate from potassium trifluoromethanecarboxylate, the steps of sulfination and oxidation according to the invention may advantageously be carried out in a single organic polar aprotic solvent, such that these steps may be carried out successively and without any intermediate step of switching between solvents, in particular in the same reactor.

Thus, the process according to the invention advantageously enables a gain in time, and hence a reduction in the cost price, due to the reduction in the number of steps necessary to obtain potassium trifluoromethanesulfonate (and triflic acid), for example.

Moreover, linking the steps of sulfination and oxidation in succession according to the invention in organic polar aprotic solvent medium makes it possible to minimize the degradation of the reaction stream resulting from the sulfination, which can occur during switching between solvents.

Thus, implementing the process of the invention makes it possible to improve the overall yield for the preparation of potassium trifluoromethanesulfonate (and triflic acid).

Finally, by not employing aqueous solvent, the process of the invention makes it possible to obtain triflic acid of electronic quality, having a low content of sulfates, or even not containing any sulfates.

Of course, the process of the invention is in no way limited just to the synthesis of potassium trifluoromethanesulfonate and to that of triflic acid.

Other features, variants and advantages of the process according to the invention will emerge more clearly upon reading the following description and examples, given by way of nonlimiting illustration of the invention.

Throughout the remainder of the text, the expressions “between . . . and . . . ”, “ranging from . . . to . . . ” and “varying from . . . to . . . ” are equivalent and are intended to mean that the limit values are included, unless indicated otherwise.

As specified above, the process for the preparation of an oxysulfide and fluorinated derivative of formula Ea-SO₃R (III) according to the invention involves an oxidation reaction of a compound Ea-SOOR (II) with an oxidizing agent in an organic solvent medium.

Within the meaning of the invention, “solvent” is intended to mean a compound which is liquid at its usage temperature and which is able, due to its content in the reaction medium, to dissolve a reagent.

Within the context of the oxidation reaction according to the invention, the organic solvent used is more particularly able to dissolve the compound of formula (II).

The reaction medium of the oxidation reaction according to the invention preferably does not contain aqueous solvent.

The absence of aqueous solvent does not preclude the possible presence of water, which would nonetheless not be able to dissolve the reagent due to the excessively small amount thereof.

Thus, the reaction medium may comprise a water content less than or equal to 10% by weight, in particular less than or equal to 4% by weight, or even not contain water. For example, the water content may be less than 100 ppm.

These small amounts of water may more particularly originate from the oxidizing agent employed for the oxidation reaction, for example aqueous hydrogen peroxide, and/or be formed by the oxidation reaction.

Within the meaning of the invention, “reaction medium” is intended to mean the medium in which the chemical reaction in question takes place; in the present case, the oxidation reaction. The reaction medium comprises the reaction solvent (organic solvent in the case of the oxidation reaction according to the invention) and, depending on the progression of the reaction, the reagents and/or the products of the reaction. In addition, it can comprise additives and impurities.

Within the meaning of the invention, “solvent” is intended to mean a single solvent or a mixture of solvents. The organic solvent used in the invention may be an organic solvent or a mixture of two or more organic solvents. In the case of a mixture, the solvents may be miscible or immiscible with one another.

The organic solvent is a polar aprotic solvent.

Aprotic solvent is intended to mean a solvent which, according to the Lewis theory, does not have protons to release.

As detailed in the remainder of the text, the organic solvent used for the oxidation reaction according to the invention may more particularly be the solvent used for the formation of the compound of formula (II) by sulfination starting from a compound of formula Ea-COOR (I).

It is understood that the solvent used must be sufficiently stable under the reaction conditions.

The organic solvent is polar. It is thus preferable for the polar aprotic solvent used according to the invention to have a significant dipole moment. Thus, its relative dielectric constant ε is advantageously at least equal to 5. Preferably, its dielectric constant is less than or equal to 50 and greater than or equal to 5, especially between 30 and 40. In order to determine if the organic solvent meets the dielectric constant conditions stated above, reference may be made, inter alia, to the tables of the publication: Techniques of Chemistry, II—Organic solvents—p. 536 et seq., 3^{rd} edition (1970).

In addition, it is preferable for the solvents used in the process of the invention to be capable of satisfactorily solvating the cations, which means that the solvent has certain basicity properties within the Lewis meaning. In order to determine if a solvent satisfies this requirement, its basicity is assessed by referring to the “donor number”. A polar organic solvent exhibiting a donor number of greater than 10, preferably of greater than or equal to 20, is chosen. The upper limit does not exhibit any critical nature. Preferably, an organic solvent having a donor number of between 10 and 30 is chosen. It should be recalled that the term “donor number”, denoted DN in abbreviation, gives an indication as to the nucleophilic nature of the solvent and reveals its ability to donate its lone pair. The definition of the “donor number” is found in the publication by Christian Reichardt, [Solvents and Solvent Effects in Organic Chemistry—VCH, p. 19 (1990)], where it is defined as the negative (−ΔH) of the enthalpy (kcal/mol) of the interaction between the solvent and antimony pentachloride in a dilute dichloroethane solution.

According to the present invention the polar solvent or solvents do not have acidic hydrogen; in particular when the polar nature of the solvent or solvents is obtained by the presence of electron-withdrawing groups, it is desirable for there not to be any hydrogen on the atom in the a position with respect to the electron-withdrawing functional group.

More generally, it is preferable for the pKa corresponding to the first acidity of the solvent to be at least equal to approximately 20 (“approximately” emphasizing that only the first figure is significant), advantageously at least equal to approximately 25 and preferably between 25 and 35.

The acidic nature can also be expressed by the acceptor number AN of the solvent, as defined by Christian Reichardt, [“Solvents and Solvent Effects in Organic Chemistry”, 2^{nd} edition, VCH (RFA), 1990, pages 23-24]. Advantageously, this acceptor number AN is less than 20 and in particular less than 18.

According to a particularly preferred embodiment, the organic solvent is of amide type. Among the amides, amides having a specific nature, such as tetrasubstituted ureas and monosubstituted lactams, are also included. The amides are preferably substituted (disubstituted for the ordinary amides).

The organic solvent may more particularly be selected from N,N-dimethylformamide (DMF), N,N-diethylformamide (DEF), N,N-dimethylacetamide (DMAC), derivatives of pyrrolidone such as N-methylpyrrolidone (NMP) and the mixtures thereof.

Another particularly advantageous category of solvents is composed of ethers, whether they are symmetrical or asymmetrical and whether they are open or closed. The various glycol ether derivatives, such as the various glymes, for example diglyme, should be incorporated in the category of the ethers.

According to a particularly preferred embodiment, the organic solvent used for the oxidation reaction according to the invention is DMF.

The oxidizing agent may be selected from peroxides, peracids, and salts thereof. For example, the oxidizing agent may be selected from aqueous hydrogen peroxide; percarbonates, especially sodium or potassium percarbonate; persulfates, especially potassium persulfate; persulfuric acid, for example Caro's salt; and organic peroxides, for example hydrogen peroxide-urea.

The oxidizing agent may be miscible or immiscible in the reaction medium. Thus, the reaction medium may be heterogeneous or homogeneous.

According to one particularly advantageous embodiment, the oxidizing agent is anhydrous.

According to another particular embodiment, the oxidizing agent is aqueous hydrogen peroxide. The aqueous hydrogen peroxide may have a concentration in water of between 10% and 80%, preferably between 30% and 70%.

Moreover, the oxidizing agent may be selected from gaseous agents, for example from the group consisting of air, oxygen, (O₂), ozone (O₃) and nitrous oxide (N₂O). Oxidation with these agents may optionally be carried out in the presence of a metal catalyst.

In accordance with the process of the invention, at least one compound of formula Ea-SOOR (II) is reacted with an oxidizing agent.

Said compound of formula (II) may be a fluorosulfinic acid (R represents a hydrogen atom in the abovementioned formula (II)), a salt of fluorosulfinic acid (R represents a monovalent cation in the abovementioned formula (II)), or an ester of fluorosulfinic acid (R represents an alkyl group in the abovementioned formula (II), in particular an alkyl group having from 1 to 10 carbon atoms).

The result thereof is thus, respectively, the preparation according to the process of the invention of fluorosulfonic acid (R represents a hydrogen atom in the abovementioned formula (III)), a salt of fluorosulfonic acid (R represents a monovalent cation in the abovementioned formula (III)), or an ester of fluorosulfonic acid (R represents an alkyl group in the abovementioned formula (III), in particular an alkyl group having from 1 to 10 carbon atoms).

According to a particularly preferred embodiment, said compound of formula (II) is a salt of fluorosulfinic acid in which R represents a monovalent cation advantageously selected from alkali metal cations, quaternary ammonium cations and quaternary phosphonium cations.

The quaternary ammonium or phosphonium cations may more preferentially be selected from tetraalkylammonium or -phosphonium, trialkylbenzylammonium or -phosphonium or tetraarylammonium or -phosphonium, the alkyl groups of which, which are identical or different, represent a linear or branched alkyl chain having from 4 to 12 carbon atoms, preferably from 4 to 6 carbon atoms, and the aryl group of which is advantageously a phenyl group. Preferably, it is the tetrabutylphosphonium cation.

According to a particularly preferred embodiment, R represents an alkali metal cation, in particular selected from sodium, potassium, cesium and rubidium cations.

According to a particular embodiment, R is the potassium cation.

As indicated above, the Ea group may represent a fluorine atom or a group having from 1 to 10 carbon atoms, selected from fluoroalkyls, perfluoroalkyls and fluoroalkenyls.

Within the context of the invention:
alkyl is intended to mean a linear or branched hydrocarbon-based chain preferably comprising from 1 to 10 carbon atoms, in particular from 1 to 4 carbon atoms;
fluoroalkyl is intended to mean a group formed from a linear or branched C₁-C₁₀ hydrocarbon-based chain comprising at least one fluorine atom;
perfluoroalkyl is intended to mean a group formed from a linear or branched C₁-C₁₀ chain comprising only fluorine atoms in addition to the carbon atoms, and devoid of hydrogen atoms;
fluoroalkenyl is intended to mean a group formed from a linear or branched C₁-C₁₀ hydrocarbon-based chain comprising at least one fluorine atom and comprising at least one double bond.

The Ea group is preferably selected from a fluorine atom and a group having from 1 to 5 carbon atoms, selected from fluoroalkyls, perfluoroalkyls and fluoroalkenyls.

According to a particularly preferred embodiment, the group Ea in the compound of formula (II) is selected from a fluorine atom, the CH₂F radical, the CHF₂ radical, the C₂F₅ radical and the CF₃ radical. The result thereof is thus, respectively, the preparation according to the process of the invention of F—SO₃R, CH₂F—SO₃R, CHF₂—SO₃R, C₂F₅—SO₃R and CF₃—SO₃R, where R is as defined above.

According to a particular embodiment, Ea represents the CF₃ radical.

It is understood that the abovementioned definitions for the groups R and Ea, respectively, may be combined.

Thus, according to a variant embodiment, the process according to the invention uses a compound of formula Ea-SOOR (II), in which:
Ea is selected from a fluorine atom, the CH₂F radical, the CHF₂ radical and the CF₃ radical; in particular, Ea is the CF₃ radical; and
R represents an alkali metal cation, preferably the potassium cation.

The process of the invention may more particularly be implemented for the preparation of a trifluoromethylsulfonate alkali metal salt (CF₃SO₃R with R representing an alkali metal cation), in particular potassium trifluoromethylsulfonate (CF₃SO₃K, or potassium triflate), which may advantageously be used to give triflic acid (CF₃SO₃H) or triflic anhydride ((CF₃SO₂)₂O), as detailed in the subsequent text.

Those skilled in the art are able to adapt the conditions for carrying out the oxidation reaction in the organic solvent in order to give the desired oxysulfide and fluorinated derivative of formula (III). In the process according to the invention, the compound of formula (II) is brought into contact with an oxidizing agent under conditions conducive to the formation of the derivative of formula (III).

The compound of formula (II) may be brought into contact with the oxidizing agent continuously, semi-continuously or batchwise. They are preferably brought into contact semi-continuously (semi-batchwise). In the case of a semi-continuous process, the oxidizing agent may be introduced continuously into the reaction medium.

The process according to the invention may be carried out in an apparatus enabling semi-continuous or continuous operation, for example in a perfectly stirred reactor, a cascade of perfectly stirred reactors advantageously fitted with a jacket, or a tubular reactor fitted with a jacket in which a heat-exchange fluid is circulating.

According to one semi-continuous implementation mode, the oxidizing agent, for example the aqueous hydrogen peroxide, may be added continuously in a liquid medium, prepared beforehand, comprising said compound of formula (II) in the organic solvent.

Generally, the concentration of compound of formula (II) in the organic solvent within the initial reaction medium is between 1% and 40% by weight, in particular between 5% and 30% by weight.

The oxidation reaction according to the process of the invention may be carried out by bringing the reaction medium to a temperature of between 20° C. and the boiling point of the organic solvent, in particular between 40° C. and 140° C. Advantageously, the oxidizing agent may be added after having pre-heated the liquid medium comprising the compound of formula (II) in the organic solvent.

The duration of the heating may be adjusted as a function of the reaction temperature chosen. It may be between 30 minutes and 24 hours, in particular between 1 hour and 20 hours, and more particularly between 2 hours and 7 hours.

The progression of the oxidation reaction may advantageously be monitored by an analytical method.

The progression of the oxidation reaction, for example the concentration of compound of formula (II), may be monitored in-line (via a sampling loop, for example) or in situ by Raman spectrometry, by near infrared spectrometry or by UV spectroscopy, preferably by Raman spectrometry.

Within the context of monitoring the state of progression of the reaction by Raman spectrometry, the reaction within which the oxidation reaction takes place may be fitted with a Raman probe, connected by an optical fiber to the Raman spectrometer, said probe making it possible for example to monitor the concentration of compound of formula (II) in the medium.

The compound of formula Ea-SOOR (II) used for the oxidation reaction according to the process of the invention may be prepared beforehand from the reaction, in the presence of an organic solvent, of a compound of formula Ea-COOR (I), in which Ea and R are as defined above, with a sulfur oxide (sulfination reaction).

Thus, as mentioned above, it is possible, according to the invention, to link the steps of sulfination and oxidation in succession, within the same organic solvent, without requiring an operation for changing the solvent.

According to another of its aspects, the present invention relates to a process for the preparation of an oxysulfide and fluorinated derivative of formula (III):

Ea-SO₃R (III)

with:
Ea representing a fluorine atom or a group having from 1 to 10 carbon atoms, selected from fluoroalkyls, perfluoroalkyls and fluoroalkenyls; and
R representing hydrogen, a monovalent cation or an alkyl group;
   comprising at least the consecutive steps consisting in:

(i) bringing into contact, in the presence of an organic polar aprotic solvent, a compound of formula Ea-COOR (I) with a sulfur oxide, in order to obtain a compound of formula Ea-SOOR (II); and

(ii) adding, to the reaction mixture obtained at the end of step (i) of sulfination, an oxidizing agent, in order to obtain the derivative of formula (III).

The organic solvent may be more particularly as defined above. It may preferably be N,N-dimethylformamide (DMF).

The reaction medium of steps (i) and (ii) preferably comprises a water content less than or equal to 10% by weight, in particular less than or equal to 4% by weight, or even does not contain water.

As indicated above, the small amounts of water of the reaction medium originate from the oxidizing agent in the case of a hydrated oxidizing agent such as aqueous hydrogen peroxide, or from the water produced by oxidation-reduction during the oxidation reaction.

The sulfination reaction is known and already described, for example, in document EP 0 735 023. Those skilled in the art are able to adjust the conditions for carrying out the step (i) of sulfination. In the process according to the invention, the compound of formula (I) is brought into contact with a sulfur oxide under conditions conducive to the formation of the derivative of formula (II).

According to preferred conditions for carrying out the step (i) of sulfination of the process of the invention, it is desirable to control the content of impurities present in the reaction medium.

More specifically, the content of labile hydrogen atoms of the sulfination reaction medium (step (i)), or more exactly of releasable protons borne by its various components, including their impurities, should be less than the content of fluorinated groups released by the decomposition of the compound of formula (I). The term “labile hydrogen atom” or “releasable proton” is understood to mean a hydrogen atom which is capable of being pulled off in the form of a proton, by a strong base. In practice, they are the protons of acidic functional groups which have a pKa of less than approximately 20. The lower the content of releasable protons, the lower the risk of side reactions and the better the sulfination yield. The content of releasable protons which are present in the medium is at most equal to 20% of the initial concentration of said compound of formula (I). Advantageously, this content is at most equal to 10%, preferably to 1% (in moles), with respect to the initial content of compound of formula (I).

The main molecule bearing labile hydrogen atoms is generally water, which is capable of releasing up to two protons per molecule. Generally, it is preferable to use dehydrated reagents and solvents, so that the content by weight of water of each of the reagents is at most equal to 1 per 1000, relative to the total weight of said reagent. Depending on the combined reaction conditions, such water contents may be satisfactory but, in some cases, it may be advantageous to operate at lower levels, for example of the order of 1 per 10 000. However, it is not necessarily essential to remove all of the water and a water/compound of formula (I) molar ratio of strictly less than 10%, preferably less than 1%, may be tolerated.

Furthermore, it is desirable for metal impurities to be in small amounts. Metal elements can be present as impurities introduced especially by the reagents, the solvent or else by the metal equipment as a result of corrosion. Thus, in order not to introduce additional metal contamination, it is important, in particular when the compound of formula (I) is a salt of fluorocarboxylic acid, for the latter to be prepared by reaction of a base with the corresponding fluorocarboxylic acid under conditions such that the base is introduced in an amount in the vicinity of within ±5% and preferably equal to the stoichiometric amount. More generally, it may be indicated that the two categories of metals which may be essentially present, namely transition elements having two valency states (such as copper, iron or chromium) and the elements of group VIII (in particular metals of the platinum group, which is the cluster consisting of platinum, osmium, iridium, palladium, rhodium and ruthenium), have to be present in the medium at a content, expressed relative to the fluorocarboxylic acid, at most equal to 1000 molar ppm, preferably at most equal to 10 molar ppm.

The compound of formula Ea-COOR (I) used in step (i) may be completely or partially a recycled compound which can be obtained, for example, by separation at the end of the oxidation reaction or which can originate from a subsequent synthesis step, for example by separation at the end of the preparation of a fluorinated derivative of sulfonic acid, or of a fluorinated compound having a sulfonic acid anhydride functional group, as detailed in the subsequent text.

When the compound of formula Ea-COOR (I) used in step (i) is a salt, that is to say when R represents a monovalent cation, said salt may have been obtained by salification of the corresponding acid, that is to say the compound of formula Ea-COOR (I) in which R represents a hydrogen atom. According to a particular embodiment, when the compound of formula (I) is an alkali metal salt of trifluorocarboxylic acid, in particular potassium trifluoroacetate, the latter may have been obtained by salification of the corresponding trifluorocarboxylic acid, in particular of trifluoroacetic acid. The salification agent may conventionally be selected from inorganic or organic bases, especially from hydroxides, carbonates and alkoxides of a monovalent cation. The monovalent cation may advantageously be selected from alkali metal cations, in particular sodium, potassium, cesium and rubidium, more particularly potassium. The base may preferably be selected from the group consisting of potassium hydroxide and sodium hydroxide, and it is very preferably potassium hydroxide.

The acid and the salification agent may be mixed according to any means known to those skilled in the art. A mixing device may be appropriately selected from different classes of mixers, for example stirred reactors, reactors with external recirculation loops, and dynamic mixers. According to a preferred embodiment, an intensified mixing system may be used. The mixing means may preferentially be selected from impinging jet mixers, coaxial nozzle injectors and Venturi tubes, optionally supplemented with static mixers of Sulzer or Kenics type. The intensified mixing process advantageously makes it possible to continuously and effectively bring the reagents into contact. The reaction volume may be minimized while intensifying the mixing conditions. Evacuation of the enthalpy of reaction is accelerated, which makes it possible to limit the rise in temperature and enables the use of plastic materials which are more resistant to corrosion phenomena than conventional metals (stainless steel, nickel-based steels). This technology may advantageously lead to a more economical and more productive process.

The sulfur oxide may more particularly be sulfur dioxide. It is generally employed in the gaseous form. It may also be introduced in the form of a solution, in the organic solvent chosen for the reaction, at a concentration generally varying between 1% and 10% by weight, preferably between 3% and 6% by weight.

According to a particular embodiment, the step (i) of sulfination is carried out with an initial molar ratio of sulfur oxide/compound of formula (I) less than 0.4, in particular less than 0.2, and with a concentration of sulfur oxide dissolved in the reaction medium which is kept constant over the whole duration of the reaction at a value of between 0.2 and 3% by weight.

A constant concentration of sulfur oxide in the reaction medium may be maintained by a controlled and continuous addition of sulfur oxide to the reaction medium.

Within the meaning of the invention, it is suitable to interpret constant concentration as meaning that said concentration can vary by ±20%, preferably by ±10%.

The concentration of sulfur oxide dissolved in the reaction medium may be monitored by an analytical method as described previously, in particular by Raman spectrometry. The controlled addition of sulfur oxide to the reaction medium advantageously makes it possible to convert the compound of formula (I) into a compound of formula (II) while substantially penalizing the undesired chemistry related to the degradation of the compound of formula (I) by the sulfur oxide.

Generally, the concentration of the compound of formula (I) in the organic solvent within the initial reaction medium of step (i) may be between 1% and 40% by weight, in particular between 5% and 30% by weight.

The compound of formula (I) may be brought into contact with the sulfur oxide in step (i) of the process of the invention continuously or semi-continuously (or semi-batchwise). This is preferably carried out semi-continuously, in particular in an apparatus as described above for the oxidation process according to the invention.

As an example of carrying this out semi-continuously, all the compound of formula (I) may be introduced into the organic solvent, then the sulfur oxide is added continuously.

The sulfur oxide is preferably added after having preheated the solution, formed of the organic solvent and of the compound of formula (I), to a temperature of between 50° C. and 150° C.

According to a particular embodiment, silica is introduced into the reaction medium, preferentially in an amount such that it represents from 0.1 to 10% by weight, preferably from 0.5 to 10% by weight in the reaction medium. The silica is particularly added to the solution formed of the organic solvent and of the compound of formula (I) when the process according to the invention is carried out semi-continuously. The addition of silica makes it possible to substantially reduce the corrosive impact on the reactor of the fluorides generated in the medium by the implementation of the sulfination step according to the invention.

The sulfination reaction according to step (i) of the process of the invention may be carried out by bringing the reaction medium to a temperature of between 100° C. and 200° C., in particular between 120° C. and 160° C. The sulfination reaction is advantageously carried out at atmospheric pressure but higher pressures can also be used. Thus, an absolute total pressure selected between 1 and 20 bar and preferably between 1 and 3 bar may be suitable.

According to another embodiment, the reaction can be carried out at a pressure below atmospheric pressure. The absolute total pressure can be between 1 mbar and 999 mbar, in particular between 500 mbar and 950 mbar and more particularly between 800 mbar and 900 mbar.

The duration of the heating may be adjusted as a function of the reaction temperature chosen. It may be between 30 minutes and 24 hours, in particular between 1 hour and 20 hours, and more particularly between 2 hours and 7 hours.

According to the continuous embodiment, the mean residence time, which is defined as the ratio of the volume of the reaction mass to the feed flow rate, lies more particularly between 30 min and 10 hours and especially between 2 hours and 4 hours.

In order to avoid too high a degradation of the compound of formula (II) formed at the end of the sulfination reaction, and thus to ensure good selectivity of the sulfination reaction, it may be preferable not to seek to fully convert the starting compound of formula Ea-COOR (I).

The progression of the reaction may be monitored by the degree of conversion of the compound of formula (I), which denotes the ratio of the molar amount of compound of formula (I) consumed during the reaction to the total amount of compound of formula (I) in the initial reaction medium. This degree may be readily calculated after assay of said compound of formula (I) remaining in the reaction medium.

The step (i) of sulfination is generally carried out until a degree of conversion of said compound of formula (I) ranging from 50% to 100%, in particular from 55% to 90%, is obtained.

At the end of step (i) of sulfination, the reaction medium thus generally comprises a mixture of the compound formed, Ea-SOOR (II), and the compound Ea-COOR (I) which has not been consumed.

In a second step (ii) of the process of the invention, and consecutive to the sulfination step described above, an oxidizing agent is added to the reaction medium, in order to form, by oxidation reaction with the compound of formula Ea-SOOR (II), the desired derivative of formula Ea-SO₃R (III).

The conditions for carrying out the oxidation reaction are as described above.

The reaction medium obtained at the end of step (ii) of oxidation generally comprises a mixture of the oxysulfide and fluorinated derivative of formula Ea-SO₃R (III) and of the starting compound Ea-COOR (I) which has not been consumed. The latter may advantageously be isolated and recycled, for example used in step (i) of the process according to the invention.

According to a particularly advantageous embodiment, steps (i) and (ii) may be carried out in the same reactor in semi-continuous mode. According to another embodiment, steps (i) and (ii) may be carried out in two tubular reactors in series.

Advantageously, the process of the invention makes it possible to prepare a salt of fluorosulfonic acid starting from a salt of fluorocarboxylic acid.

More particularly, it makes it possible to obtain an alkali metal salt of trifluoromethanesulfonate (CF₃SO₃R with R representing an alkali metal cation), in particular potassium trifluoromethylsulfonate (CF₃ SO₃K, or potassium triflate).

The latter may advantageously be used to obtain triflic acid (CF₃SO₃H) or triflic anhydride ((CF₃SO₂)₂O), as detailed in the subsequent text.

Advantageously, the oxysulfide and fluorinated derivatives of formula (III) obtained according to the invention, in particular an alkali metal salt of trifluoromethylsulfonate (CF₃SO₃R, with R representing an alkali metal cation), may be used for the preparation of fluorinated derivatives of sulfonic acid, in particular trifluoromethanesulfonic acid, more commonly referred to as triflic acid (CF₃SO₃H).

Thus, according to yet another of its aspects, a subject of the invention is a process for preparing a fluorinated derivative of sulfonic acid of formula (IV)

Ea-SO₃H (IV)

Ea representing a fluorine atom or a group having from 1 to 10 carbon atoms, selected from fluoroalkyls, perfluoroalkyls and fluoroalkenyls; in particular, Ea representing the CF₃ radical;
comprising at least the following steps:
preparation, according to the process described above, of an oxysulfide and fluorinated derivative of formula Ea-SO₃R (III), in which R represents a monovalent cation or an alkyl group, in particular an alkali metal cation, in an organic solvent S1; and
acidification of the compound of formula (III) in order to obtain the desired fluorinated derivative of sulfonic acid of formula (IV).

In particular, a fluorinated derivative of sulfonic acid of formula Ea-SO₃H, in which Ea is as defined above, may be prepared according to the invention via at least the following steps:

(a1) bringing into contact, in the presence of an organic solvent S1, a compound of formula Ea-COOR (I), in which R represents a monovalent cation or an alkyl group, in particular an alkali metal cation, with a sulfur oxide, in order to obtain a compound of formula Ea-SOOR (II);

(b1) adding, to the reaction mixture obtained at the end of step (a1) of sulfination, an oxidizing agent, in order to obtain an oxysulfide and fluorinated derivative of formula Ea-SO₃R (III); and

(c1) acidification of the compound of formula (III) in order to obtain the desired fluorinated derivative of sulfonic acid of formula (IV).

Advantageously, the process of the invention is carried out in order to prepare trifluoromethanesulfonic acid (Ea represents the CF₃ radical).

According to a particular embodiment, the compound of formula (I) used in step (a1) is an alkali metal salt of trifluorocarboxylic acid, in particular potassium trifluoroacetate (CF₃COOK), and leads, at the end of step (c1), to trifluoromethanesulfonic acid (CF₃SO₃H).

As described above, the conversion of the carboxyl compound of formula (I) during the sulfination reaction (step (a1)) is generally not total.

The acidification of the mixture of the compounds of formula Ea-SO₃R and Ea-COOR leads to the mixture of the desired fluorinated derivative of sulfonic acid Ea-SO₃H and fluorocarboxylic acid Ea-COOH, for example to the mixture of triflic acid and trifluoroacetic acid (Ea represents CF₃).

The fluorinated derivative of sulfonic acid Ea-SO₃H may be isolated from the mixture obtained at the end of the acidification, for example by distillation.

The fluorinated derivative of carboxylic acid Ea-COOH is advantageously recycled, for example in the process according to the invention.

The steps of sulfination (a1) and oxidation (b1) are more particularly carried out under the conditions described above.

The acidification of the compound of formula Ea-SO₃R (III) (more generally, of the mixture thereof with the unreacted carboxyl compound Ea-COOR (I)) may be carried out as detailed below.

According to a first alternative, the acidification is carried out via the steps consisting in:

(1) substituting the organic solvent S1, and if present the water, from the reaction mixture comprising said oxysulfide and fluorinated derivative of formula (III) (and generally the unreacted carboxyl compound of formula Ea-COOR (I)) by an organic solvent S2; said solvent S2 being inert with regard to the acidification agent, immiscible with the solvent S1 and having a boiling point greater than that of the solvent S1 and/or forming an azeotrope with the latter; and

(2) acidifying the mixture formed at the end of step (1), comprising the derivative of formula (III) (and generally the unreacted carboxyl compound of formula Ea-COOR (I)) in said solvent S2, in order to obtain the desired fluorinated derivative of sulfonic acid Ea-SO₃H (IV) (generally, in a mixture with the fluorocarboxylic acid Ea-COOH).

The organic solvent S1 may be substituted by the solvent S2 by the following consecutive steps:
elimination of the majority of the organic solvent S1, and, if present, of the water, by distillation;
addition of the organic solvent S2; and
elimination of the residual solvent S1 by azeotropic distillation.

As seen above, the organic solvent S1 is preferably N,N-dimethylformamide (DMF).

The organic solvent S2, which has a higher boiling point than DMF, may for example be selected from high boiling point alkanes, for example decalin (including the mixture of isomers), and aromatic derivatives bearing an electron-withdrawing group, for example ortho-dichlorobenzene (ODCB) or nitrobenzene.

The acidification of the compound of formula Ea-SO₃R (III) (and of the unreacted carboxyl compound Ea-COOR (I)) in step (2) may be carried out by addition of sulfuric acid, in particular in oleum form, to the liquid mixture obtained at the end of step (1).

The sulfuric phase may then be extracted from the mixture obtained by separation of the phases after acidification, and the fluorinated derivative of sulfonic acid of formula (IV) may be isolated, for example by distillation of the sulfuric phase.

The solvent S2 may advantageously be recycled, for example in step (1).

The fluorinated derivative of carboxylic acid Ea-COOH is advantageously recovered in order to be recycled, for example in the process according to the invention.

According to a second alternative, the acidification step may be carried out via the steps consisting in:

(1′) adding, to the reaction mixture comprising said oxysulfide and fluorinated derivative of formula (III) (and generally the unreacted carboxyl compound of formula Ea-COOR (I)) in the organic solvent S1, a solvent S2′ which is unable to dissolve the compound of formula (III), in an amount conducive to the precipitation of the compound of formula (III) from the mixture of solvents S1/S2′;

(2′) isolating the solid precipitated at the end of step (1′) formed of the compound of formula Ea-SO₃R (III) (and generally of the unreacted carboxyl compound of formula Ea-COOR (I)); and

(3′) acidifying the solid recovered at the end of step (2′), in order to obtain the desired fluorinated derivative of sulfonic acid Ea-SO₃H (IV) (generally in a mixture with the acid Ea-COOH).

The organic solvent S1 is preferably N,N-dimethylformamide (DMF).

The S1/S2′ mixture may be a homogeneous or heterogeneous mixture, preferably a homogeneous mixture. The S2′ may in particular be an alkane, an aromatic derivative, for example ortho-dichlorobenzene (ODCB) or toluene, a halogenated derivative, for example dichloromethane, an ether or an ester.

The acidification of the solid in step (3′) may be carried out by addition of sulfuric acid or oleum.

As described above, the fluorinated derivative of sulfonic acid of formula (IV) may then be isolated, for example by distillation of the sulfuric phase.

The fluorinated derivative of carboxylic acid Ea-COOH is advantageously recovered in order to be recycled, for example in the process according to the invention.

The fluorinated derivative of sulfonic acid Ea-SO₃H obtained according to the invention may advantageously be converted into an anhydride of formula (Ea-SO₂)₂O (V).

In particular, the triflic acid obtained according to the invention may be used to obtain trifluoromethanesulfonic acid of formula (CF₃—SO₂)₂O (triflic anhydride).

Thus, according to yet another of its aspects, a subject of the invention is a process for the preparation of an anhydride compound of formula (Ea-SO₂)₂O (V), Ea representing a fluorine atom or a group having from 1 to 10 carbon atoms, selected from fluoroalkyls, perfluoroalkyls and fluoroalkenyls; in particular, Ea representing the CF₃ radical;

comprising at least the following steps:
preparation, according to the process described above, of a fluorinated derivative of sulfonic acid of formula Ea-SO₃H; and
anhydrization of the derivative of formula Ea-SO₃H in order to obtain said desired anhydride compound of formula (V).

In particular, an anhydride compound of formula (Ea-SO₂)₂O (V), in which Ea is as defined above, may be prepared according to the invention via at least the following steps:

(a2) bringing into contact, in the presence of an organic solvent S1, a compound of formula Ea-COOR (I), in which R represents a hydrogen atom, a monovalent cation or an alkyl group, in particular an alkali metal cation, with a sulfur oxide, in order to obtain a compound of formula Ea-SOOR (II);

(b2) adding, to the reaction mixture obtained at the end of step (i) of sulfination, an oxidizing agent, in order to obtain an oxysulfide and fluorinated derivative of formula Ea-SO₃R (III);

(c2) in the case in which R is different from a hydrogen atom, acidification of the compound of formula (III) in order to obtain the fluorinated derivative of sulfonic acid Ea-SO₃H; and

(d2) the anhydrization of the compound of formula Ea-SO₃H in order to form said desired anhydride compound of formula (V).

Advantageously, the process of the invention is carried out in order to prepare trifluoromethanesulfonic anhydride (Ea represents the CF₃ radical).

According to a particular embodiment, the compound of formula (I) used in step (a2) is an alkali metal salt of trifluorocarboxylic acid, in particular potassium trifluoroacetate (CF₃COOK), and leads, at the end of step (d2), to trifluoromethanesulfonic anhydride ((CF₃—SO₂)₂O).

The steps of sulfination (a2) and oxidation (b2), and optionally acidification (c2), are more particularly carried out under the conditions described above.

The anhydrization reaction is known to those skilled in the art and is more particularly described in the document U.S. Pat. No. 8,222,450.

The fluorinated derivatives of sulfonic acid of formula Ea-SO₃H, especially triflic acid, and the anhydride compounds of formula (Ea-SO₂)₂O, especially triflic anhydride, can be used in various applications, especially as acid catalyst, as protective group in organic synthesis, as synthon in the fields of pharmaceuticals, agrochemistry or electronics, or as salt for the electronics industry, or as component of an ionic liquid.

The invention will now be described by means of the following examples, of course given by way of nonlimiting illustration of the invention.

### EXAMPLES

The degree of conversion of a reagent corresponds to the ratio of the molar amount of reagent consumed (converted) during a reaction to the initial amount of reagent.

The product yield from a reagent corresponds to the ratio of the molar amount of product formed to the molar amount of initial reagent.

### Example 1

### Preparation of Potassium Trifluoromethylsulfonate by Oxidation of Potassium Trifluoromethylsulfinate by H₂O₂ in N,N-dimethylformamide (DMF)

### i. Preparation of Potassium Trifluoromethylsulfinate (CF₃SOOK) by Sulfination of Potassium Trifluoroacetate (CF₃COOK) in N,N-dimethylformamide (DMF)

The following are introduced at room temperature into a 500 ml jacketed reactor equipped with a condenser having an aqueous glycol solution at −15° C., with a stirrer and with baffles:
200 g of anhydrous N,N-dimethylformamide (DMF);
50 g of potassium trifluoroacetate (KTFA), i.e. a KTFA concentration equal to 20% by weight in the DMF-KTFA mixture.

The reactor is equipped with a Raman probe which makes it possible to monitor, in the medium, the concentration of dissolved SO₂; this probe is connected by an optical fiber to the Raman spectrometer.

The medium is stirred and brought to a temperature of 100° C.

Via a dip pipe connected to a pressurized sulfur dioxode cylinder, an amount of 1.25 g of gaseous SO₂ is continuously introduced into the reactor through a micrometric regulating valve, so as to have a concentration of dissolved SO₂ equal to 0.5% by weight and an initial SO₂/KTFA molar ratio of 0.059.

The temperature is brought to 145° C. while keeping the SO₂ concentration constant at 0.5% by weight. The reaction is allowed to take place for 5 hours while regulating the SO₂ concentration at 0.5% by weight.

After 5 hours, the reaction mixture is cooled and analyzed by NMR, and the results are as follows:
Degree of conversion of the potassium trifluoroacetate: 90%;
Yield of potassium trifluoromethylsulfinate: 64.8%.

### ii. Oxidation of the Potassium Trifluoromethylsulfinate by Aqueous Hydrogen Peroxide in DMF

The solution resulting from the sulfination reaction of potassium trifluoroacetate in DMF, prepared as described in point i. above, with a total weight of 267.19 g, is brought to 60° C., then an aqueous solution of aqueous hydrogen peroxide (titer by weight=30%) is added to it over three hours.

The total amount of aqueous hydrogen peroxide used is two molar equivalents relative to the content of potassium trifluoromethylsulfinate.

The medium is then maintained at 60° C. for an additional 2 hours and 51 minutes, during which monitoring by in situ Raman spectrometry makes it possible to monitor the evolution of the species.

At the end of this maintenance time, the content of residual peroxides is monitored and analysis, by ¹⁹F NMR, of an aliquot makes it possible to establish that the yield of potassium trifluoromethylsulfonate is 98.44%.

### Example 2

### Preparation of Potassium Trifluoromethanesulfonate by Oxidation of Potassium Trifluoromethanesulfinate by Sodium Percarbonate in DMF

A suspension of sodium percarbonate (20.8 g) in DMF is brought to 60° C., then a solution resulting from the sulfination reaction of potassium trifluoroacetate in DMF, prepared as described in the preceding example 1, with a total weight of 176.73 g, is added over this medium in 2-3 hours.

At the end of this maintenance time, the content of residual peroxides is monitored and analysis, by ¹⁹F NMR, of an aliquot makes it possible to establish that the yield of potassium trifluoromethylsulfonate is 90.7%.

### Example 3

### Preparation of Triflic and Trifluoroacetic Acids

The reaction medium obtained at the end of the oxidation according to the preceding example 2 is distilled under reduced pressure (160 mbar) then decalin is added to it (200 ml, mixture of isomers). The distillation is continued by means of a Dean-Stark apparatus which makes it possible to regularly draw off the distilled DMF until the boiler is exhausted. The total weight of distilled DMF is 164.1 g.

150 ml of oleum at 20% are then added, and the sulfuric phase is drawn off.

The sulfuric phase is then distilled under reduced pressure, in order to lead to 9.4 g of pure trifluoroacetic acid (CF₃COOH) and 17.6 g of pure triflic acid (CF₃SO₃H), respectively.

1. A process for the preparation of an oxysulfide and fluorinated derivative of formula (III)
Ea-SO₃R (III)
comprising bringing into contact, in the presence of an organic polar aprotic solvent, a compound of formula (II)
Ea-SOOR (II)
Ea representing a fluorine atom or a group having from 1 to 10 carbon atoms, selected from fluoroalkyls, perfluoroalkyls and fluoroalkenyls; and
R representing hydrogen, a monovalent cation or an alkyl group;
with an oxidizing agent.

2. The process as claimed in claim 1, in which the reaction medium does not contain aqueous solvent.

3. The process as claimed in claim 1, in which the reaction medium comprises a water content less than or equal to 10% by weight.

4. The process as claimed in claim 1, in which said organic polar aprotic solvent is an amide type solvent.

5. The process as claimed in claim 4, in which said organic polar aprotic solvent is selected from the group consisting of N,N-dimethylformamide (DMF), N,N-diethylformamide (DEF), N-methylpyrrolidone (NMP) or N,N-dimethylacetamide (DMAC).

6. The process as claimed in claim 5, in which said organic polar aprotic solvent is N,N-dimethylformamide (DMF).

7. The process as claimed in claim 1, in which said oxidizing agent is selected from aqueous hydrogen peroxide; percarbonates; persulfates; and hydrogen peroxide-urea.

8. The process as claimed in claim 1, in which R represents a monovalent cation selected from alkali metal cations, quaternary ammonium and quaternary phosphonium cations.

9. The process as claimed in claim 1, in which Ea is selected from a fluorine atom, the CH₂F radical, the CHF₂ radical, the C₂F₅ radical and the CF₃ radical.

10. The process as claimed in claim 1, in which the progression of the oxidation reaction is monitored in-line or in situ by Raman spectrometry, by near infrared spectrometry or by UV spectroscopy.

11. The process as claimed in claim 1, for the preparation of a trifluoromethylsulfonate alkali metal salt.

12. A process for the preparation of an oxysulfide and fluorinated derivative of formula (III)
Ea-SO₃R (III)
with:
Ea representing a fluorine atom or a group having from 1 to 10 carbon atoms, selected from fluoroalkyls, perfluoroalkyls and fluoroalkenyls; and
R representing hydrogen, a monovalent cation or an alkyl group;
comprising at least the consecutive steps of:
(i) bringing into contact, in the presence of an organic polar aprotic solvent, a compound of formula Ea-COOR (I) with a sulfur oxide, in order to obtain a compound of formula Ea-SOOR (II); and
(ii) adding, to the reaction mixture obtained at the end of step (i) of sulfination, an oxidizing agent, in order to obtain the derivative of formula (III).

13. The process as claimed in claim 12, in which the reaction medium of steps (i) and (ii) comprises a water content less than or equal to 10% by weight.

14.-21. (canceled)

22. A process for the preparation of a fluorinated derivative of sulfonic acid of formula (IV)
Ea-SO₃H (IV)
Ea representing a fluorine atom or a group having from 1 to 10 carbon atoms, selected from fluoroalkyls, perfluoroalkyls and fluoroalkenyls;
comprising at least the following steps:
preparation of an oxysulfide and fluorinated derivative of formula Ea-SO₃R (III), R representing a monovalent cation or an alkyl group, in an organic solvent S1 according to the process of claim 1; and
acidification of the compound of formula (III) in order to obtain the desired fluorinated derivative of sulfonic acid of formula (IV).

23.-28. (canceled)

29. The process as claimed in claim 22, for the preparation of trifluoromethanesulfonic acid.

30. A process for the preparation of an anhydride compound of formula (V)
(Ea-SO₂)₂O (V)
Ea representing a fluorine atom or a group having from 1 to 10 carbon atoms, selected from fluoroalkyls, perfluoroalkyls and fluoroalkenyls;
comprising at least the following steps:
preparation of a fluorinated derivative of sulfonic acid of formula Ea-SO₃H according to the process of claim 1; and
anhydrization of the compound of formula Ea-SO₃H in order to obtain said desired anhydride compound of formula (V).

31. The process as claimed in claim 30, for the preparation of trifluoromethanesulfonic anhydride.

32. The process as claimed in claim 7, in which said oxidizing agent is sodium or potassium percarbonate.

33. The process as claimed in claim 7, in which said oxidizing agent is potassium persulfate.

34. The process as claimed in claim 8, in which R represents an alkali metal cation.
